(19) United States
(12) Patent Application Publication
(10) Pub. No.: US 2026/0114798 A1
(43) Pub. Date: Apr. 30, 2026

(54) APPARATUS AND METHOD FOR DETERMINING A PARAMETER OF SKIN IN A REGION OF INTEREST OF A PERSON' S BODY

(71) Applicant: Robert KATAMAY, Oberwil (CH)

(72) Inventor: Robert KATAMAY, Oberwil (CH)

(21) Appl. No.: 19/156,328

(22) PCT Filed: Feb. 13, 2023
(86) PCT No.: PCT/EP2023/053495

§ 371 Date: Aug. 13, 2025

(51) Int. Cl. A61B 5/00; A61B 17/00; A61B 17/3209; A61B 18/20; A61B 34/10; A61B 90/00

(52) U.S. Cl. CPC A61B 5/442; A61B 5/0077; A61B 5/742; A61B 17/32093; A61B 18/20; A61B 34/10; A61B 90/36; A61B 2017/00747; A61B 2034/104; A61B 2034/107; A61B 2090/366

(57) ABSTRACT

An apparatus is introduced for determining a parameter of skin in a region of interest of a person's body. The apparatus comprises a camera (1) arranged and configured to record images of the region of interest. A control unit (3) of the apparatus is configured to determine the parameter of the skin in the region of interest by analyzing one or more images of the region of interest recorded by the camera (1) while the region of interest is manipulated.

### TECHNICAL FIELD

The invention relates to an apparatus and to a method for determining a parameter of skin in a region of interest of a person's body.

### BACKGROUND ART

The aesthetic result of plastic surgery as well as curative surgery in a patient's face strongly depends on the skills of the surgeon. Plastic surgery is considered to include the disciplines aesthetic surgery, reconstructive surgery and burn surgery, while curative surgery is considered to include tumor surgery, i.a. Parameters of the skin often affect the post-op aesthetic result of any such surgery as well as other anatomic properties of the face. Traditionally, the surgeon manually examines the skin upfront to any planned surgery in order to finally define a cutting pattern. The evaluation of the skin as well as the determination of a cutting pattern heavily depends on the experience and knowledge of the surgeon. The definition of the cutting pattern in turn heavily impacts the aesthetic result of such surgical intervention.

### DISCLOSURE OF THE INVENTION

It is a general object of the invention to provide assistance means for supporting a surgeon in planning and/or preparing a surgical intervention.

According to a first aspect of the present invention, an apparatus is provided for determining a parameter of skin in a region of interest of a person's body. The apparatus comprises a camera arranged and configured to record images of the region of interest. And the apparatus comprises a control unit configured to determine the parameter of the skin in the region of interest by analyzing one or more images of the region of interest recorded by the camera while the region of interest is manipulated.

As a result, the preparation and/or planning of a surgical intervention not only relies on the skills of the surgeon, but is at least partially conducted by means of an apparatus. This allows a more standardized assessment of parameters of the person's/patient's skin finally leading to a more predictable aesthetic result of the surgery. The data gained by such semi- or fully automated inspection by means of the apparatus can further be used for simulating a future aesthetic result and demonstrating the simulated result to the person prior to the surgery. However, not only the planning but also the evaluation whether to conduct a surgical intervention or not is supported by the present invention. For example, a demonstration of the future aesthetic result may lead to a decision between the surgeon and the person to abstain from the envisaged surgical intervention.

Accordingly, images taken by the camera while the person's skin in the region of interest is manipulated contribute to an analysis of the person's skin in the region of interest. The one or more images are analyzed by the control unit, preferably by a computer vision and/or pattern recognition software executed by and/or run on the control unit. This constitutes a completely new approach for analyzing the parameter by way of support of computerized means, thereby allowing a more standardized categorization of skin properties.

The apparatus at minimum includes the control unit e.g. embodied in a computer such as a laptop, and the camera. For enhanced inspection, the camera preferably is a 3D camera external to the computer but connected thereto. The camera may be attached to a stand which stand then is part of the apparatus and can be considered as an examination stand for inspecting the person.

The purpose of the apparatus as well as of the later introduced method is to investigate a parameter of a person's skin in the region of interest. The knowledge of the parameter can be used later on for different purposes. The parameter can be used for preparing surgery in the region of interest and help the surgeon to better assess the skin properties. The parameter can be used in determining a cutting pattern in the region of interest. Such cutting pattern in turn may be used in a computer simulation anticipating the surgery along the cutting pattern and demonstrating the simulated aesthetic result, and/or in a computerized controlled automated cutting along the cutting pattern. Different cutting patterns may be proposed and lead to different results in the simulation, which results may help the person and/or the surgeon to conclude about the preferred cutting pattern. The result of the proposed method may be used in plastic surgery and/or curing surgery. The parameter as a result of the determination may also be used in cosmetic advice, and or in advice for other skin treatment.

In a very preferred embodiment, the parameter is elasticity of the skin, and hence, in particular elasticity of the skin in the region of interest. Elasticity in the present context may either denote a result of deformation in response to the manipulation relative to some defined standard deformation. Hence, elasticity may be measured in form of an index. In an alternative, the one or more of the elastic moduli, i.e. Young modulus, shear modulus, bulk modulus are determined, e.g. in units of [Pa]. Hence, it is preferred to investigate the skin in the region of interest that is the same region that later on will be treated in surgery or any other way. In particular, in case of surgical intervention, the surgeon is interested in learning about the parameters of the skin in the region of the intervention. The present apparatus allows a more standardized examination of such parameter/s.

Elasticity of the skin preferably can be supported by manipulating the skin. Manipulation is preferably defined as to physically impact the skin, i.e. to exert a force to the skin and thereby dislocate the skin from its state absent such force. Such manipulation in particular is applied to an area of excessive skin potentially to be removed. The manipulation may be performed by means of a manipulation tool as described in detail below, or may be performed manually by a person, such as the surgeon, other medical personnel, or other support personnel. It is preferred that the manipulation includes one or more of shifting, stretching, tightening, twisting and pushing together the skin in the region of interest, preferably in different directions.

In another embodiment, the parameter of the skin is a location of adhesion. In some regions, the skin is not too rigidly attached to the underlying tissue and rather is relocatable, while in other regions or spots, collectively referred to as locations, the skin may be fixedly attached to the underlying tissue, bone, etc. and be hardly relocatable. The latter locations can be identified in response to a manipulation of the skin. For an upcoming surgery, it is also of interest to the surgeon to learn about any such locations of adhesion in the region of interest which is the region that will be surgically treated. Again, the present apparatus allows a more standardized examination of such parameter/s.

Such locations of adhesion, also referred to as anchorage locations, can also be identified manually or by means of a manipulation tool, in particular thereby dislocating the skin. Again, the manipulation preferably includes one or more of shifting, stretching, tightening, twisting and pushing together the skin in the region of interest, preferably in different directions. For example, given a constant force of manipulation, the skin may be dislocated farther in case the manipulation engages in a region of the skin far from an anchorage location of the skin. In turn, the skin dislocates less in case the manipulation engages in a region of the skin close to or at an anchorage location. In such scenario, a direction of manipulation may contribute to the determination of anchorage locations. Adhesion/anchorage locations of the skin may also be determined in addition to the elasticity of the skin.

The region of interest may be any region of a person's body desired to be cut for plastic or other surgery, and any subsequent surgery hence may apply to any such region of the body. However, in a preferred embodiment, a particular region of interest is the face or a specific sub-area of the face of a person, or the neck of a person. In such areas of the person's skin, a deviation of the cutting of only 1 mm may have significant impact on the aesthetic result. While facial expression is significant for a person's appearance and the area of skin in the person's face is limited, the skin in the face and/or neck and its elasticity may heavily depend on the underlying tissue and its elasticity. The tissue may include bones, muscles, fat, etc. In addition, the elasticity of the skin may also depend on the adhesion of the skin to the underlying tissue, which e.g. in the region of the eyelid is low, while in the region of the cheek is rather high.

In a very preferred embodiment, the region of interest includes an eyelid of a person's face. Eyelid surgery is popular for many years. In eyelid surgery, excessive portions of skin are removed by surgical intervention in order to tighten the eyelid. In extreme cases, excessive portion of skin may impact the field of visibility. In other cases, eyelid surgery is conducted for aesthetic reasons. Preferably, the region of interest is represented by the upper eyelid. However, also the lower eyelid may represent the region of interest in another approach. By means of surgical intervention at the lower eyelid, lacrimal sacs may be removed, again by removing the excessive portion of skin from the lower eyelid.

In a different embodiment, the region of interest may include one or more of the cheek, a region between the cheek and the ear, and the region behind the ear. Cutting patterns in one of these regions of interest are preferred for face lifting.

In other embodiments, the region of interest is defined by an area that contains either a malignant mark in the skin to be removed, such as a black cancer spot, or a benign mark, such as a birthmark or a liver spot.

As indicated above, the apparatus may preferably comprise a manipulation tool arranged and configured to manipulate the person's skin in the region of interest. It is preferred that the manipulation tool effects one or more of shifting, stretching, tightening, twisting and pushing together the skin in the region of interest. Preferably, the manipulation tools allows to manipulate in some of the above ways. Preferably, the manipulation tool is configured and arranged to one of shift, stretch, tighten, twist and push together the skin in different directions. The manipulation tool preferably manipulates the skin in the region of interest in an automated manner. The usage of the manipulation tool that typically is electrically driven also allows for a more standardized manipulation of the person's skin. In addition, the inspection of the parameter of the skin can be conducted without any contact between the person and the surgeon or other examining personnel.

The manipulation tool preferably is connected to the control unit via an interface, such as USB, and preferably is controlled by the control unit.

In a preferred embodiment the manipulation tool comprises a mechanical structure for in-contact manipulating the person's skin in the region of interest. Such mechanical structure may e.g. be a pin or other mechanical structure, such as a swab, tweezers, or the like. In a very preferred embodiment, the manipulation tool comprises an air blower for generating an air jet directed at the region of interest for manipulating the person's skin. The air blower is a simple means to contactless manipulate the person's skin. The air blower preferably comprises a fan that is driven by an electromotor. The fan may reside in a housing and sucks in ambient air from one open end of the housing. Preferably, a nozzle is attached to the other end of the housing for shaping and expelling the air jet. For the present application, it is in particular preferred to generate an air jet of small diameter in order to apply a focused air jet to the region of interest. Accordingly, it is preferred that a diameter of the nozzle is 0.5 cm or less. With such small diameter and an appropriate distance between the nozzle and the region of interest, it is possible to even stimulate different locations within the region of interest.

In a preferred embodiment, the manipulation tool can be operated at different activation levels. This may be achieved by applying different power levels to the drive of the manipulation tool. As a result, the manipulation tool may be configured to exert different force levels onto the region of interest for manipulation purposes. In case of an air blower, the control unit may be configured to cause the air blower to eject air at different pressure forces. The parameter may then be determined by the control unit subject to the manipulation of the skin in response to applying the different activation levels.

The camera preferably is a 3D camera comprising at least two 2D cameras spaced apart and recording 2D images, and a processor configured to calculate a 3D image from the recorded two 2D images. By means of a 3D camera, the operator/surgeon not only may receive a planar 2D image, but receives information about the third dimension in addition. The processor may be internal in the camera, or, alternatively, external to the camera, and even may be represented by the control unit.

The camera as well as the manipulation tool, if any, are electrically connected to the control unit. The control unit may be embodied by a computer, e.g. a laptop. The electrical connection may be embodied wirebound or wireless. Via a suitable interface, such as USBx in case of wirebound communication, or IEEE 802.11 (WLAN) for wireless communication, images taken by the camera are transmitted to the control unit. As to the manipulation tool, the control unit preferably is configured to activate the manipulation tool via a suitable interface, e.g. one of the ones indicated above. In a first embodiment, the camera is triggered by the control unit to provide digital data in form of digital images to the control unit, while the manipulation tool is controlled by the control unit to be switched on and off.

In a preferred embodiment, the control unit is configured to determine the parameter of the skin in the region of interest not only by analyzing the one or more images of the region of interest recorded by the camera while the region of interest is manipulated, preferably by the manipulation tool, although possible. Instead, one or more images of the region of interest taken while the skin in this region is not manipulated are also analyzed. These images are also referred to as reference images. They allow to better assess the effect of the manipulation of the skin. The one or more reference images can either be supplied from elsewhere to the control unit and be stored there. This includes the one or more reference images being taken with a camera different than the camera of the apparatus. On the other hand, it is preferred that the one or more reference images are taken by the camera of the apparatus. Then, resolution, aperture and other parameters of the camera are the same for all images, preferably also the position of the camera. Regardless where the reference images come from, the control unit preferably performs a comparison between the one or more images recorded by the camera with one or more reference images.

In this embodiment, the region of interest is recorded while being manipulated, and while not being manipulated. All of the resulting images are taken by the camera of the apparatus. The one or more images recorded while the skin is manipulated (in short recorded images, irrespective if one or more) can be taken prior to the one or more reference images (in short reference images, irrespective if one or more) are taken, or after. In addition, images may be recorded for different kinds of manipulation, in particular for different activation levels of manipulation and/or for different directions of manipulation. While the scenes for the recorded images and the reference images distinguish by the state of manipulation, it is noted that additional images may be recorded for different states of facial expression of the person. For example, in case the region of interest is the upper eyelid and the parameter to be determined is elasticity of the skin of the upper eyelid, it is preferred that the recorded images as well as the reference images are taken while the person keeps the eye/s shut, since it may render difficult to evaluate the elasticity of manipulated skin when the eye/s are open and the skin of the eyelid may already be folded. More generally, the state of facial expression may include one or more defined states. The person may alter between states by way of activating muscles, for example.

The recording of several images, be it in the manipulated state or non-manipulated state, may also encompass the recording of a video.

When it comes to the comparison of the reference images with the recorded images by means of a computerized approach representing a comparison between the non-manipulated state and the manipulated state of the skin, it is preferred to have the control unit to identify one or more features in the reference images, and identify the same one or more features in the recorded images. The identification of features as well as the comparison may be achieved by computer vison or pattern recognition software executed by the control unit. In a different sequence, features are identified first in the recorded images and thereafter searched for in the reference images.

A feature is regarded as a characteristic in the image that can computationally be detected e.g. based on a change of color, a 3D elevation or recess and/or based on one or more other parameters. In real life, a feature in the face of the person may correspond to an anatomic characteristic such as the eyebrow, the eyelid gap, contours of the lids, in particular the lower contour of the upper lid, the upper contour of the lower lid, or the lid fold. Further features may not be regular features of the anatomy of the eye and/or the face, but may be individual features of the person, such as a birthmark or a liver spot. Any feature in the region of interest, which is detectable by means of pattern recognition in one of the recorded images and the reference images may be of interest for the analysis.

In the above example of the eyelid as region of interest major features extend horizontally, if straight or curved. For manipulating such features, the skin preferably is manipulated in vertical direction, i.e. the manipulation tool preferably applies a force having at least some vertical component. In case the manipulation tool is an air jet it may preferably be directed at the region of interest with an angle such that the skin is moved either to the top or to the bottom in vertical direction, and even more preferably subsequently to the top and to the bottom, or vice versa. Hence, it is less preferred to direct the air jet straight i.e. orthogonal to the region of interest. Generally, it is preferred to manipulate features that essentially extend horizontally in a vertical direction, and/or to manipulate features that essentially extend vertically in a horizontal direction. Features mainly extending in the vertical direction may include wrinkles close to the outer corner of the eye.

In a next step, the control unit preferably is configured to determine a distance between the one or more features (collectively “features” in the following) identified in the reference images and the same features identified in the recorded images. For this purpose, the control unit may first align and scale the reference images with/to the recorded images e.g. by image processing, and thereby assure that the sections compared with each other are of same size, resolution, brightness, etc. at least in a tolerable range, which facilitates the identification of the features and the subsequent tracking of the features in the other images. This also enables using a common scale for identifying the position of the features in the images and hence to determine a distance between the positions of the features according to this scale. Accordingly, a distance is determined between the position of a feature in the non-manipulated state and a position of the same feature in the manipulated state.

In a next step, it is preferred that the control unit is configured to determine the parameter subject to the determined distance/s. For example, the larger the distance between the features is the higher the elasticity of the skin is in this region of interest. The smaller the distance is, the lower the elasticity of the skin is. In case more than one feature is identified to be dislocated in the same direction in response to the manipulation, an average distance may be calculated and be taken as or contribute to the measure for the elasticity of the skin in the region of interest.

While in the above embodiment, focus is on the features displaced in response to the manipulation, other features may contribute to the determination of the parameter, too. For example, a feature may not necessarily be dislocated in response to the manipulation. And/or a feature may disappear in response to the manipulation, at least in the recorded images. And/or a features may appear from new in response to the manipulation.

Preferably, any such classes of features may contribute to the determination of the parameter, and preferably each class is weighted in its contribution to the parameter. This may be implemented by a formula. In one embodiment, the formula may look like:

$$ε = w 1 * {F}_{gr 1} + w 2 * {F}_{gr 2} + w 3 * {F}_{gr 3} + w 4 * {F}_{gr 4}$$

wherein:
ε is the elasticity or an index representing the elasticity of the skin;
F_{grx} are features of the same class, wherein
gr1 is the class of dislocated features;
gr2 is the class of not dislocated features;
gr3 is the class of newly appearing features;
gr4 is the class of disappearing features;
w1 to w4 are weighting factors.

The features of F_{gr1} may each individually be determined. For example, for gr1, Fgr1 may represent the average distance for all dislocated features. For example, for gr2, Fgr2 may represent the number of not dislocated features. For example, for gr3 and gr4, the corresponding Fgr3 and Fgr4 may represent the number of newly appearing or disappearing features respectively. By adjusting the weights w1 to w4, it may be determined what impact the classes have on the measure of elasticity. For example, the dislocated feature class Fri may be weighted most, i.e. w1>w2, w3, w4.

While the determination of the parameter so far is dependent on the analysis of the recorded images and, possibly, the reference images, additional input and corresponding images can be taken into account. E.g., in case the manipulation tool is configured to act at different activation levels, images recorded at different activation levels are taken into account when determining the parameter. The higher the activation level of the manipulation tool is, the bigger dislocation of a feature can be expected, and vice versa.

In case of an air blower, a distance between the nozzle and the region of interest, specifically the spot the air jet meets the region of interest, may be relevant for the determination of the parameter. For example, the smaller the distance is the bigger the displacement of a feature can be expected. In turn, the farer the distance is, the lower displacement can be expected. In a preferred embodiment, a distance sensor is provided for measuring such distance, e.g. for measuring the distance between the nozzle of the air blower and the skin.

Other data may contribute to the determination of the parameter, in particular personal data including one or more of age of the person, gender of the person and origin of the person. In case of the parameter being elasticity, there can be a relation between the elasticity and the age and/or the gender and/or the origin of the person. Such data may be collected upfront and supplied to the control unit to be available for the calculation of the parameter.

While in the above embodiments, the determination of the parameter is conducted computerized by means of an algorithm implemented in a software the control unit executes after the recorded images and possibly the reference images are available, other steps may also be conducted in a computerized, automatic manner, such as:

The camera is triggered to record the reference images of the region of interest while the manipulation tool is deactivated, e.g. in response to a deactivation of the manipulation tool, or at the beginning of the examining method. For example, once the person to be examined is in a reference position with respect to the camera and the manipulation tool, the operator may trigger a program for determining the parameter, which in a first step activates the manipulation tool to manipulate the skin in the region of interest. In response to the activation, e.g. straight on or with a defined delay, the camera may be controlled to record the images during manipulation. In response to the images being recorded, the manipulation tool automatically is deactivated. In response to deactivating the manipulation tool, the reference images are recorded by the camera and supplied to the control unit. In response to receiving the reference images, the control unit may automatically start determining the parameter.

As to the hardware equipment of the apparatus, preferably a stand is provided wherein the camera and/or the manipulation tool are mounted to the stand. The stand may be a set-up comparable to a stand for conducting ophthalmologic investigations at the ophthalmologist, where different devices can be swung into position in front of a person sitting on a chair. Accordingly, the stand may include a chair or a bed for the person to sit or to lie on and take an examining position facing the camera and the manipulation tool. The camera and/or the manipulation tool are preferably mounted to a rack in a position allowing the person to record or manipulate when in an examining position.

The manipulation tool preferably is slidable mounted to the stand or rack. In particular in case of the manipulation tool being an air blower, the air jet is allowed to adjust to persons of different heights by taking different positions. Preferably, the position of the air blower is electrically adjustable and the control unit is configured to position the air blower at a defined position.

In addition, or alternatively, the manipulation tool, e.g. the air blower, is pivot mounted to the stand allowing the air blower to be tilted. The tilt may then be electrically adjustable, in particular wherein the control unit is configured to position the air blower in a defined tilted position which allows the skin to be pushed up, or down.

All the above findings may also be true for the camera: The camera may be slidable mounted to the stand or rack allowing the camera to adjust to persons of different heights. Preferably, the position of the camera is electrically adjustable, wherein the control unit is configured to position the camera in a defined position. It is also preferred to position the camera at a defined distance with respect to the face of the person, and more preferably with respect to the region of interest. In addition, it is preferred that the one or more images are recorded with a standardized background light that allows good illumination of the scene and the region of interest respectively.

This above positioning step/s may be conducted prior to the recording of the images. For doing so, the control unit preferably controls the camera in one or more of: Positioning the camera; recording the one or more images. And, the control unit preferably controls the manipulation tool in one or more of: Positioning the manipulation tool; activating the manipulation tool with a defined power; switching the manipulation tool between different activation levels.

In a different embodiment of the hardware equipment of the apparatus, the apparatus may include an operating table that is equipped with the camera and/or the manipulation tool, which is either mounted directly to the table, or mounted elsewhere but aligned towards the table.

Preferably, the apparatus comprises an output unit for outputting the determined parameter. The output unit may be a display for illustrating the determined parameter. In particular, the control unit can be configured to automatically display the parameter once determined. Moreover, the display may be controlled by the control unit to present the recorded images and/or the reference images. In such embodiment, the determined parameter may be color-coded displayed on the display, in particular color-coded in the region of interest in the recorded displayed images and/or the displayed reference images.

In a further embodiment, the parameter serves as a basis to generate a cutting pattern with respect to the region of interest for a surgical intervention, possibly in combination with other parameters. In one embodiment, the cutting pattern is generated from scratch by means of the control unit.

In order to generate a person specific cutting pattern it is preferred that a model of the region of interest of the person is generated preferably by means of the camera and the control unit. In a simple approach, the reference image may serve as model. And/or, a 3D scan may be performed by the camera which results in a 3D model at least of the region of interest, which serves as a basis for coordinates also describing the cutting pattern to be generated or selected.

In a different embodiment, an existing cutting pattern may be suggested based on at least the determined parameter. One or more existing cutting patterns, which can be regarded as standard cutting patterns, are preferably stored in the control unit or a database of the surgeon or hospital and/or elsewhere remote such as in the cloud, at least accessible for the control unit. The cutting patterns of such set may constitute standard cutting patterns suggested for different values of the at least one parameter determined. The stored standard cutting patterns may also vary subject to other parameters, such as eye width, etc. The width of the eye may e.g. be determined by the distance between the commissura medialis palpebrarum and the commissura lateralis palpebrarum.

A standard cutting pattern of the set may be selected in an automated manner by means of the control unit or triggered by the control unit to be selected from a database or from the cloud. In such scenario, the control unit may input the determined parameter as well as possibly one or more other parameters such as geometric dimensions in the region of interest, and/or age, gender, etc. Then, a standard cutting pattern of the set is automatically selected that is assigned to the input parameters.

In possible next step, the selected or preselected cutting pattern/s may be modified in view of the determined parameter. E.g. individual cuts of the cutting pattern may be extended and/or reduced in length, and/or individual cuts of the cutting pattern may be shifted with respect to other cut/s of the cutting pattern and/or with respect to one or more features of the person in the region of interest. In case of adapting an existing cutting pattern, the control unit may preferably computerized generate the adapted cutting pattern from the existing cutting pattern, and/or may computerized suggest more than one adapted cutting pattern for the surgeon or the person to select from.

In case the region of interest is the eyelid, the cutting pattern may comprise of at least two cuts, an upper cutting line and a lower cutting line. However, additional cutting lines may be envisaged. In the embodiment of computer-supported generating a cutting pattern dependent on the determined parameter, the program executed by the control unit or in the cloud may, for example, suggest or set the upper cutting line, determine and set a number of additional cutting lines if any, and suggest and set a lower cutting lines. The setting of a cutting line preferably includes its unique definition, e.g. by means of coordinates, preferably 3D coordinates, etc.

E.g., the upper cutting line and/or the lower cutting line of the selected or preselected cutting pattern may be lifted in view of a high elasticity as determined. Instead, the upper cutting line and/or the lower cutting line of the standard cutting pattern may be lowered in view of a low elasticity as determined, all in the vertical dimension.

Preferably, the control unit is configured to simulate the facial expression, at least in the region of interest, in view of the generated and/or selected cutting pattern, which allows the aesthetic result to be demonstrated to the person. Once the cutting pattern is determined, the control unit is configured to apply image editing routines that simulate, based one or more of the reference images taken absent any manipulation of the skin, the effect of a hypothetical surgery with the determined cutting pattern. Image editing routines as such are known and may be applied and or adjusted to the specific purpose. The edited image preferably is displayed to the person and provides an idea of the aesthetic facial expression after surgery with the respective cutting pattern. Such simulation may be conducted for different states of facial expressions such as with closed eye/s and with open eye/s. Accordingly two images taken from the person with the two different facial expressions are edited and simulate the person after surgery with open eyes as well as with closed eyes. The open eyes edited image in particular is useful to demonstrate the lid gap resulting from surgery. The surgery typically impacts the lid gap, i.e. the opening of the eye, whereas the lid gap has impact on the overall facial expression (tired/attentive). Accordingly, such simulation may further provide assistance to the person and/or the surgeon to assess the effect of the surgery.

Preferably, any simulated state represented by an edited image can be 3D viewed by the patient in view of the preferred 3D property of the images recorded. Preferably, the person may, in the GUI of the display, modify and/or shift the individual cutting lines and immediately get the aesthetic result shown on the screen. In such cases, it may be preferred that the program defines a possible cutting lines in response to the determined elasticity, such that only cutting patterns are offered for selection that fit to the determined elasticity.

In another embodiment, the selection and/or determination of a cutting pattern is supported by an ANN (artificial neural network), hence an artificial intelligence component, preferably provided in the cloud, i.e. remote from the apparatus.

Machine learning algorithms as included in the ANN build a model based on sample data, known as training data, in order to make predictions or decisions for new input vectors. Accordingly, the model built from the training data is configured to perform accurately on new, unseen input data after having experienced training data.

In such embodiment, the ANN preferably comprises a supervised learning algorithm that is trained by supplying training data in combination with an output associate with such training data. Such supervised learning algorithm may include one of support-vector machines, linear regression, logistic regression, naïve Bayes, linear discriminant analysis, similarity learning decision trees, K-nearest neighbor algorithm, neural networks.

Such ANN preferably is fed/input with a pre-op image of the person, at least of the region of interest. Such pre-op image can be taken by the camera of the apparatus. Preferably, the pre-op image is a 3D image. For example, the one or more reference images taken for the determination of the parameter can be used. Images of different scenes of the person can be used such as—in the case of eye lid surgery—with eyes open, eyes closed, etc. In addition to such image, a cutting pattern is used as input for the ANN. Additional parameters can be used as input, such as gender, age, etc. In response to such input, the ANN provides an output in form of an envisaged post-op result, preferably in form of an image depicting the person if surgery along the cutting pattern were performed on that person.

Such ANN preferably is trained by corresponding input—output training data representing training examples, where surgery including a defined cutting pattern was performed on a person resulting in a post-op appearance of the region of interest that was affected by the surgery. Hence, the training data taken from a training example includes an input vector Vᵢ={ref. pre-op image; cutting pattern; . . . } to which input vector Vᵢ an output is assigned comprising at least the post-op image of the person (again, at least of the region of interest). For training the ANN, it is preferred that as many surgeons or hospitals as possible supply pre-op images and post-op images to the ANN in combination with the cutting pattern applied during the surgery. The training results in the learning algorithm to build a model that can predict output data, presently the envisaged visual appearance of the region of interest of the person after surgery was conducted with the cutting pattern indicated.

Preferably, the control unit is configured to connect to and make use of the ANN and/or to train such ANN by supplying training data including input vectors and the corresponding output. The latter can only be performed after surgery given that the result of the surgery represents the output data. The former can be used in preparation of the surgery, e.g. by providing an input vector of the patient to the ANN containing the person's image, and one or more parameters determined, and/or other parameters. As additional input the surgeon defines or selects a cutting pattern to be applied to the patient. The ANN may return the envisaged post-op result, i.e. the appearance of the region of interest of the person after surgery. By submitting different cutting patterns, different anticipated surgery results may be displayed to the patient, in order to choose the most preferred one.

Cutting patterns themselves can also be stored in a hospital or general database of cutting patterns e.g. in the cloud. In such database, preferred and/or individual cutting patterns may be stored and even tagged by the individual surgeon with preferences of certain cutting patterns. Such database may also hold cutting patterns based on fashion trends, such as ones leading to short or high lid folds.

Preferably, in the ANN visual feature extraction is applied. Features are extracted from the input image/s. For example, the extracted vectors that represent a part of the input vector Vᵢ are m-dimensional vectors, where, preferably, m≥32 (e.g., m=64 or 128) and is typically less than or equal to 512 or 1024. The ANN extraction give rise to vectors having a small memory footprint. The extraction can be constrained to make sure that all vectors are normalized to a same reference length, hence allowing more meaningful comparisons between the vectors. Each vector can for instance be obtained in output of an L2 Normalization layer. Each vector is preferably a 128D vector, normalized using the L2 norm. Each of the vector component values is therefore less than or equal to 1. Other normalization schemes can be contemplated. Still, having vectors normalized all to a same length allows more meaningful comparisons.

A cutting pattern automatically generated by the program/control unit or selected by the person and/or surgeon in response to various automatically preselected suggestions by the control unit may further be used in various ways. Preferably, the apparatus comprises a cutting tool, preferably an optical cutting tool, such as a laser, most preferably a carbon dioxide (CO2) laser, that is provided to contactless cut the lines of the cutting pattern into the skin of the person, and hence performs at least the cutting according to the cutting pattern automatically with no or little intervention of the surgeon. Preferably, the cutting tool is mounted to a robot arm, which preferably is computer controlled in its movements, e.g. by way of the control unit. In this case, the control unit not only supports the determination of the cutting pattern but also supports executing or executes the cutting pattern on the patient. This requires a precise and safe bedding of the patient, of course. Such cutting by a laser has the advantage over manual cutting with a scalpel that the interaction is contact-free. In turn, when using a scalpel the bare setting of the scalpel on the skin in order to prepare the cutting may yet displace the skin and possibly lead to a deviation from the intended cutting path. In the same way, the actual cutting performed by the laser does not exert a force onto the skin. Instead, when using a scalpel, the contact cutting and the forces executed during cutting may evoke an unintentional displacement of the skin, again leading to a possible deviation from the intended cutting path.

In a different embodiment, the apparatus comprises a marking tool, preferably an optical marking tool, such as a laser, most preferably a carbon dioxide (CO2) laser, to write or burn.

In a different embodiment, the apparatus comprises a light tool configured to project the cutting pattern onto the region of interest of the person sitting in the stand. This may show the person where the envisaged cuts will take place. In a different variant, the control unit is configured to overlay the cutting pattern in the region of interest of the recorded images and/or the reference images displayed. Such overlaid image may support the surgeon in a later surgical intervention. Finally, the cutting lines may also be projected onto the region of interest before or even during the surgical intervention, to provide guidance to the surgeon.

According to a second aspect of the present invention, a use of an apparatus according to any of the preceding embodiments is provided, for preparing a surgical intervention in the region of interest of the person.

According to a third aspect of the present invention, a method is provided for determining a parameter of skin in a region of interest of a person's body. The person's skin is manipulated in the region of interest. One or more images of the region of interest are recorded while the region of interest is manipulated. And the parameter of the skin in the region of interest is computerized determined by analyzing the recorded one or more images.

Preferably, the parameter is elasticity and the region of interest includes an eyelid of the person's face.

Preferably, the manipulation of the person's skin in the region of interest is triggered computerized. This may include one or more of computerized positioning a manipulation tool, such as an air blower, and/or computerized activating the manipulation tool, such as the air blower thereby generating the air jet directed at the region of interest, thereby one or more of shifting, stretching, tightening and pushing together the skin in the region of interest.

Preferably, also the recording of the one or more images may be triggered computerized by triggering a camera to record the one or more images. In addition, the camera may positioned computerized.

In a preferred embodiment, one or more reference images of the region of interest are taken, preferably by the same camera, without the person's skin in the region of interest being manipulated, and the parameter is determined based on a comparison of the one or more reference images with the one or more recorded images.

Preferably, images are taken by the camera of different facial expressions of the person affecting the region of interest, in case the region of interest is the person's face. In case of the eyelid representing the region of interest, images may be taken with the facial expression of one or more of the person having the eye/s open, having the eye/s wide open, having the eye/s closed, having the eyes convulsively closed, having the eyebrow/s raised. Preferably, each of the facial expressions is recorded twice, once with manipulation, once without manipulation.

In another embodiment, the determination step includes identifying one or more features in the one or more reference images, identifying the one or more features in the one or more recorded images, determining a distance between the one or more features identified in the one or more reference images and the one or more features identified in the one or more recorded images, and determining the parameter subject to the determined distance. The parameter may in addition be determined subject to one or more of a defined power the manipulation tool is activated with or a defined force the manipulation tool exerts on the skin, a distance between a nozzle of the air blower and the skin in the region of interest, and personal data including one or more of age of the person, gender of the person and origin of the person. All these steps are preferably conducted computerized.

In one embodiment, the control unit may:
control the camera to take one or more reference images of the region of interest while the manipulation tool is deactivated;
activate the manipulation tool to manipulate the skin in the region of interest;
control the camera to record the one or more images of the region of interest while the manipulation tool is activated, and
determine the parameter by comparing the one or more reference images with the one or more recorded images.

The determined parameter may then be displayed, in particular in combination with the recorded images and/or the reference images, e.g. by color-coding the region of interest therein by the parameter.

Preferably, a cutting pattern is generated computerized for the region of interest and is based at least on the determined parameter, and possibly further parameters. The cutting pattern may be generated from scratch, or may be generated by adapting an existing cutting pattern dependent on at least the parameter. Such cutting pattern may be used either in the preparation of a surgical intervention only, or may additionally be used for the actual surgery, i.e. the cutting, along the cutting pattern into the skin of the person in the region of interest, by means of a computer controlled cutting tool, preferably by means of a computer controlled laser.

Such cutting pattern may be projected onto the region of interest of the person by means of a light tool; or it may be displayed on the recorded images and/or the reference images.

According to a fourth aspect of the present invention, a computer program element is provided comprising computer program code means configured to perform a method according to one of the embodiments of the method when executed on a control unit.

### BRIEF DESCRIPTION OF THE DRAWINGS

The invention will be better understood and objects other than those set forth above will become apparent when consideration is given to the following detailed description of embodiments of the invention. Such description makes reference to the annexed drawings, wherein:

FIG. 1 shows a schematic apparatus and a scene representing a method, both according to embodiments of the present invention;

FIG. 2 shows a block diagram of an apparatus according to an embodiment of the present invention;

FIG. 3 shows sketches of various states of the human eye, which sketches at the same time may represent images recorded during the method, according to an embodiment of the present invention;

FIG. 4 illustrates a flow chart representing a method according to an embodiment of the present invention; and

FIGS. 5 and 6 illustrates flowcharts representing variants of a method according to embodiments of the present invention.

### MODES FOR CARRYING OUT THE INVENTION

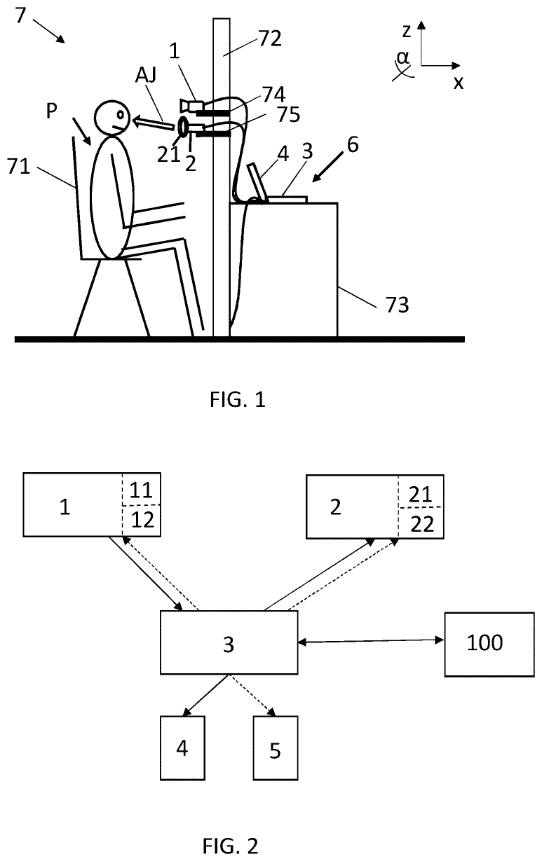

FIG. 1 shows a schematic apparatus and a scene representing a method, both according to embodiments of the present invention. The arrangement comprises a stand 7 as may be installed in an examination center, a doctor's office, a hospital, or any other office. The stand 7 is provided for inspecting parameters of a person's skin.

In particular, the stand 7 comprises a chair 71 for the person/the patient to sit on, in front of a rack 72, and, at discretion, a table 73. The rack 72 supports a camera 1 and a manipulation tool 2 explained in more detail below. The camera 1 and the manipulation tool 2 each are mounted to an assigned shelf board 74, 75 of the rack 72, which shelf boards 74, 75 presently are slidable mounted to the rack 72 in z-direction.

In a different embodiment, the stand comprises a table, such as an operation table, instead of a chair, for the person/the patient to lie on. Camera 1 and/or manipulation tool 2 may then be arranged accordingly, e.g. to a rack above the patient.

The camera 1 and the manipulation tool 2 are electrically connected to a computer 6, such as a laptop. The electrical connection may be embodied wirebound, as shown in FIG. 1, or wireless. The computer 6 includes a control unit 3, typically in form of a processing unit of the computer 6, as well as a display 4. Via a suitable interface, images taken or recorded by the camera 1 are supplied to the control unit 3. The control unit 3 further is configured to activate the manipulation tool 2 via a suitable interface. Accordingly, in a first embodiment, the camera 1 provides digital data in form of digital images to the control unit 3, while the manipulation tool 2 is controlled by the control unit 3 at least to be switched on and off.

However, in other embodiments, the camera 1 may also be controlled by the control unit 3, e.g. it may be triggered to record images, and be triggered to stop recording images.

In further embodiments, the position and/or the orientation of the camera 1 and/or the manipulation tool 2 may be controllable by means of the control unit 3. For example, the shelf boards 74, 75 supporting the camera 1 and the manipulation tool 2 are electrically movable along the z-axis of the rack 72. For this purpose, each shelf board 74, 75 may be assigned an electric motor (not shown) such that via the control unit 3 the vertical position z of the shelf boards 74, 75 and hence of the camera 1 and the manipulation tool 2 may be adjusted, in particular subject to the height of a person P to be examined.

In the present scenario, a portion of the skin of the person P is desired to be inspected by means of the apparatus as illustrated. In particular, a portion of the person's skin in his/her face shall be examined, for example as elasticity. In the present example, it is assumed that the region of interest is the eyelid of the person P. Accordingly, both, the camera 1 and the manipulation tool 2 are directed at one eyelid of the person P.

The manipulation tool 2 is intended to manipulate the skin of the person P in the region of interest. Manipulation shall include any deformation of the skin in the region of interest, including tensioning or pushing the skin in the region of interest. The effect of such manipulation then is recorded by the camera 1. Presently, the manipulation tool 2 is embodied as an air blower. The air blower has a housing including a fan, and a nozzle 21 in the housing or attached to the housing for allowing the air to exit and/or be focused. The air blower hence generates an air jet AJ directed at the region of interest in the person's face. In FIG. 1, the air jet AJ is schematically indicated by an arrow. During manipulation, the skin in the region or sub-region of interest is tensioned and/or folded. Absent a controllable manipulation tool, the manipulation may be conducted manually by a responsible person.

The deformation serves as an indicator of the elasticity of the person's skin in the region of interest, which may vary from person to person. The deformation is recorded by the camera 1 in form of one or more images, or even as a video, which is sent to the control unit 3 for evaluation. The control unit 3 may then analyze the one or more recorded images, and may automatically derive the elasticity of the person's skin in the region of interest. The value of the elasticity may then be displayed to the operator on the display 4 of the computer 6, for example.

In a further embodiment, one or both of the camera 1 and the manipulation tool 2 are pivot mounted to the rack 72 of the stand 7. In addition to be adjustable vertically, the devices 1 and 2 are then tiltable in a vertical plane x-z, e.g. by angle alpha. This may lead to an even better positioning of the camera 1 and/or the manipulation tool 2 with respect to the region of interest. Again, such tilting may be controllable by means of the control unit 3.

FIG. 2 illustrates a block diagram of an apparatus according to an embodiment of the present invention. The control unit 3 is interconnected with the camera 1 and the manipulation tool 2. Presently, the straight arrow between the camera 1 and the control unit 3 represents the transmission of one or more images recorded by the camera 1 to the control unit 3. The dashed arrow in opposite direction indicates an optional control of the operation and/or the position of the camera 1 by means of the control unit 3. This may include controlling a trigger 11 of the camera 1 to take one or more images. And/or this may include controlling the position of the camera relative to the rack 72, e.g. by means of an electric motor 12, which presently is assigned to the camera 1 while in practice it may rather be assigned to a shelf board supporting the camera 1.

The straight arrow between the manipulation tool 2 and the control unit 3 represents the transmission of control signals such as ON and OFF for activating and deactivating the manipulation tool 2. In this example, the actuator in the manipulation tool 2 may, for example, be a fan 21 of an air blower. The dashed arrow between the control unit 3 and the manipulation tool 2 indicates an optional control of the positioning of the manipulation tool 2 by means of the control unit 3. This may include controlling the position of the manipulation tool 2 relative to the rack 72, e.g. by means of an electric motor 22, which presently is assigned to the manipulation tool 2 while in practice it may rather be assigned to a shelf board supporting the manipulation tool 2.

FIG. 2 also shows a sample artificial neural network 100 remote from the apparatus, in the cloud. The double arrow indicates that the control unit 3 may request for envisaged surgery results in response to providing an image of the region of interest and an envisaged cutting pattern. The double arrow additionally indicates, the in a preferred embodiment, the surgeon or hospital may also train the ANN 100 by supplying training data in form of pre-op image/s and cutting patterns, and corresponding post-op image/s.

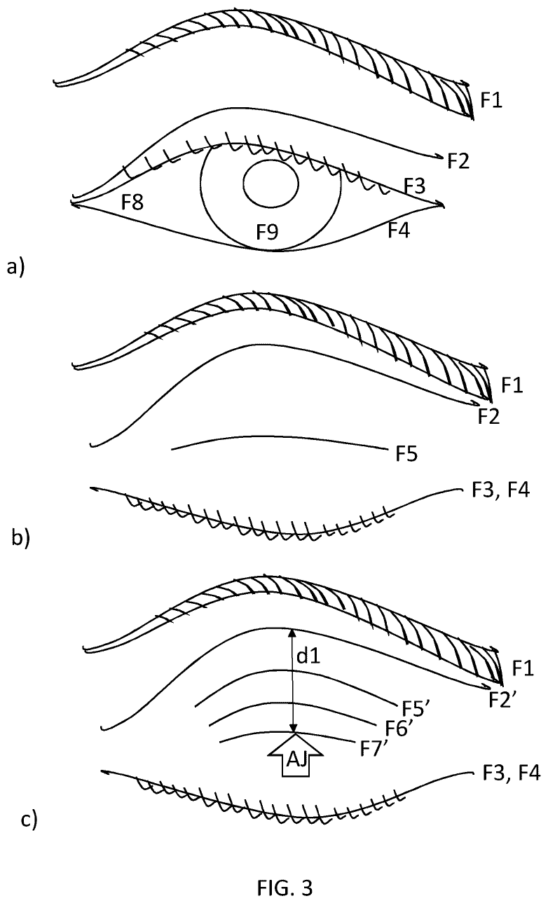

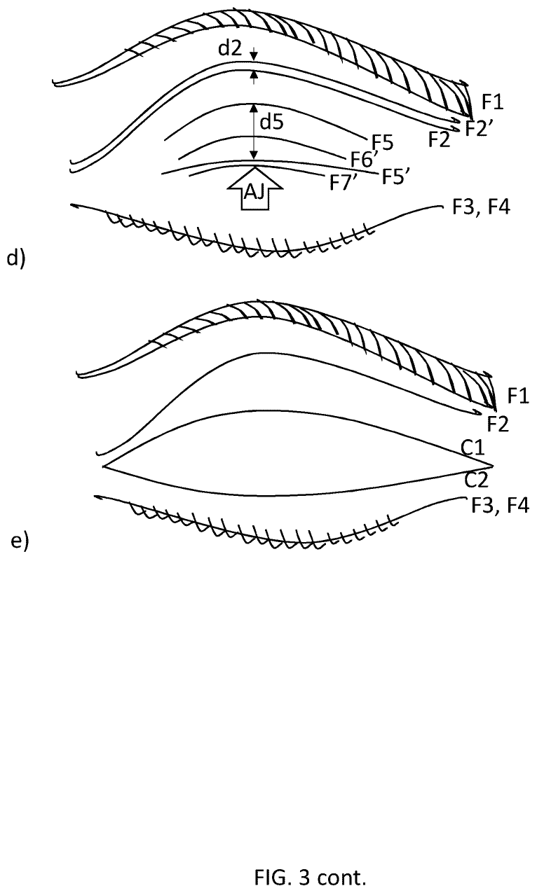

FIG. 3 shows sketches of various states of the human eye, which sketches at the same time may represent images recorded during the process, according to an embodiment of the present invention.

FIG. 3a) illustrates a sketch of the human eye with the upper eyelid F3 open. The upper eyelid F3 and the lower eyelid F4, and more precisely the lower contour of the upper eyelid F3 and the upper contour of the lower eyelid F4 define the eyelid gap F8 and surround the eye as such including its vitreous body. The upper eyelid F3 carries eyelashes indicated in FIG. 3a); the lower eyelid F4 does so, too, however not illustrated. F9 indicates the iris of the eye surrounding the pupil. At the top, the eyebrow F1 is illustrated. Between the eyebrow F1 and the upper eyelid F3—and more precise the lower contour of the upper eyelid F3, the eyelid fold is indicated by F2.

FIG. 3b) illustrates a sketch of the human eye with the facial expression of the upper eyelid F3 being closed, i.e. iris F6 and pupil are covered by the upper eyelid F3, which, in this state meets the upper contour of the lower eyelid F4 in a simplified approach. Between the eyebrow F1 and the lower contour of the upper eyelid F3, the eyelid fold F2 may still be visible, while, just as an example, another fold F5 may become visible which may be invisible/covered by the upper eyelid in the open eye's state according to FIG. 3a).

Generally, what is illustrated schematically as anatomy of the human eye may be interpreted as features Fx when it comes to computer vision or pattern recognition when investigating an image of the human eye. E.g. each of the contours indicated by F1, F2, F3, F4, F5 may be a feature to be computationally detected e.g. based on a change of color, a 3D elevation or recess and/or based on one or more other parameters the recorded image is analyzed for.

The present features Fx are basically all curves mainly extending horizontally owing to the anatomy of the human eye. In addition, one or more of these features F1 to F5 may change their horizontal position z in response to the person moving the eyelids by contraction of corresponding muscles.

Further features may not be regular features of the anatomy of the eye and/or the face, but may be individual features of the person, such as a birthmark or a liver spot. Any feature in the region of interest, which is detectable by means of computer vision can be used for determining the parameter.

What is shown in FIGS. 3a) and 3b) hence is not only a schematic representation of the human eye in two different states “eye open” and “eye closed”, but can also represent reference images taken by a camera or the camera 1 of FIG. 1.

FIG. 3c) illustrates the same person's region of interest in a different state. In this state, the person holds the eye closed, while an external manipulation tool in form of an air blower generates and directs an air jet AJ to the closed upper eyelid. In response to the manipulation of the upper eye lid, some of the features Fx identified in the non-manipulated state of the closed eye according to FIG. 3b) may be dislocated, and/or some may remain at the same position, and/or some may disappear, and/or some new features may appear and be newly detected in response to the manipulation. In the particular example of FIG. 3c), the features F1, F3 and F4 remain at the more or less the same position in comparison with FIG. 3b). Feature F2 may slightly be dislocated along the vertical axis z into position shown as F2′, feature F5 is strongly dislocated to feature F5′, and new features F6′ and F7′ may appear in response to the manipulation. E.g., the fold or contour F7′ may appear directly at the location where the air jet AJ meets the upper eyelid, and another fold or contour F6′ may appear between F5′ and F7′.

FIG. 3d) illustrates an overlay of the two images of FIGS. 3b) and 3c), and hence an overlay of the images of the closed eye recorded absent manipulation and the closed eye recorded under impact of manipulation of the upper eyelid. For the two dislocated features F2′ and F5′, vertical distances d2 and d5 can be determined with respect to their original position on the z-axis (F2 and F5 in FIG. 3b)). Presently, the vertical distances d2 and d5 are determined or measured in the vertical plane dividing the pupil. Alternatively, vertical distances can be measured at the z-peak of the respective features, or at the middle of the eye in horizontal direction, etc.

Accordingly, a comparison of features identified in images taken at different manipulation states may allow the determination of the parameter of the skin in the region of interest, here the skin of the upper eyelid. For example, the bigger the distances d2 and d5 are, the more elastic the skin proves in this region of interest. The lower the distances are, the lower the elasticity of the skin of the eyelid is.

However, other features than the dislocated ones may also contribute to the determination. In the present example, features F1, F3 and F4 remain basically unchanged in z-direction, features F6′ and F7′ appear from scratch in response to the manipulation, and other features may disappear in response to the manipulation. All such classes or groups of features may contribute to the determination of the parameter.

Within a group, the individual features may be weighed against each other given that their impact on the elasticity may depend on their location. For example, a location of a feature close to the access point of the manipulation tool is more likely to be affected by the manipulation than a feature far away. At the same time or in addition, the anatomy and hence the absolute location of a feature may have impact on its response to the manipulation: A feature that is close or tied to a muscle or to tissue underneath will need a stronger force to be dislocated than a feature in a region that only is softly tied to the underlying tissue. In addition, and/or alternatively, the manipulation force may be taken into account when determining the parameter of the skin in the region of interest.

FIG. 3e) illustrates the closed human eye without manipulation, i.e. identical to FIG. 3b), however, with two cutting lines C1 and C2 projected onto e.g. the face of the person. These cutting lines C1 and C2, and in particular their arrangement and shape/curve, and the horizontal location of their crossing points may in one embodiment be automatically determined from the measure of elasticity. The area between the cutting lines C1 and C2 may finally be skin to be removed by way of cutting the two lines C1 and C2 in a surgical intervention. When having removed the excessive skin, the remaining skin is joined at the cutting lines C1 and C2 effecting a tensioning of the skin of the upper eyelid.

In a different embodiment, the image of FIG. 3e) may represent a reference image of the person with eyes shut as in FIG. 3b) including an overlay of the cutting lines C1, C2, e.g. by means of an image editing software. The resulting image according to FIG. 3e) may then be displayed to the person and/or the surgeon for further reference.

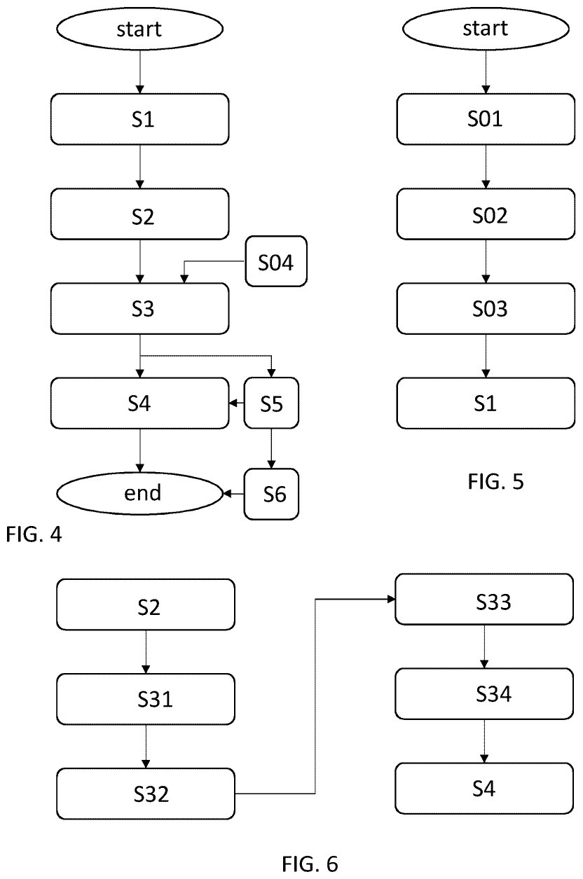

FIG. 4 illustrates a flow chart representing a method according to an embodiment of the present invention. In step “start”, the method is started. It is assumed that at the start of the method, the person/patient may have taken an examining position, e.g. in a seat or on a table, e.g. in a stand 7 as shown in FIG. 1, and may be prepared to be examined. It may be assumed that a camera and a manipulation tool are ready to go and are positioned relative to the person, and in particular relative to the region of interest of the body of the person to conduct the respective tasks explained above and below. In step S1, the person's skin is manipulated in the region of interest, preferably by means of a manipulation tool such as an air blower. Starting with the manipulation or later on, at least while the region of interest is continued to be manipulated, one or more images are recorded of the region of interest in step S2. In step S3, a parameter of the skin in the region of interest is determined at least from an analysis of the one or more images recorded from the manipulated region of interest. In optional step S4, the determined parameter is displayed on a display and shown to the person examined and/or the person conducting the examining. Then the method may “end” in this embodiment.

In a variant, in addition to or alternative to displaying the parameter in step S4, cutting lines and/or a cutting pattern may be determined in step S5 at least based on the determined parameter of skin. Such cutting lines may either be displayed on the display, e.g. in combination with a reference image of the person. In a different variant, the cutting lines may be projected onto the region of interest in step S6 of the person sitting in the stand, e.g. by means of a light beam, and/or be cut into the region of interest of the person by a computer controlled cutting tool.

Step S04 indicates a variant, in which one or reference images of a non-manipulated state of the region of interest are provided to the control unit and may be used in the analysis for determining the parameter in step S3. Such one or more digital images may be supplied to the control unit by way of file transfer, email, portable memory, etc.

FIG. 5 illustrates a flowchart representing a variant of a method according to embodiments of the present invention. In comparison to FIG. 4, additional steps are introduced after the method is started. In step S01, the manipulation tool is adjusted to a position suited for manipulating the region of interest, e.g. respecting the person's height while sitting in the stand. Preferably, such position adjustment is controlled by the control unit, i.e. the manipulation tool is shiftable mounted to the rack by means of an electric motor. Hence, the operator may move the manipulation tool up or down the rack by activating a button or control field on the computer. In an apparatus, where the manipulation tool is pivot mounted to the rack and the tilt of the manipulation tool can be electrically controlled tilted, such rotational alignment of the manipulation tool may also be included in step S01.

In an even more sophisticated system, the positioning of the manipulation tool relative to the person and in particular to the region of interest is fully automated. This may afford one or more sensors, possibly including the camera, for sensing the position of the person, and in particular for sensing or detecting the region of the interest of the person after having taken the examining position. In case the location of the region of interest is determined by means of such one or more sensors, the manipulation tool may automatically be moved and/or tilted to a position where it is enabled to manipulate the region of interest when being activated. In such embodiment, the manipulation tool is automatically positioned without human interaction.

In step S02, the same positioning is performed for the camera. The camera is adjusted to the right position for recording images from the region of interest. In the example of FIG. 1, the camera may be positioned in a vertical position of the rack that fits for the particular person, and e.g. is adjusted to the person's height while sitting in the stand. The operator may move the manipulation tool up or down the rack by activating a button or control field on the computer. In an apparatus, where the camera is pivot mounted to the rack and the tilt of the camera can electrically be controlled, e.g. by means of an electrical motor controllable by the control unit, such alignment of the camera may also be included in step S01.

In an even more sophisticated system, the positioning of the camera relative to the person and in particular to the region of interest in the person's face is fully automated. This includes one or more sensors, in particular including the camera, for sensing the position of the person, and in particular for sensing or detecting the region of the interest of the person after having taken the examining position. In case the location of the region of interest is determined by means of such one or more sensors, the camera may automatically be moved and/or tilted to a position where it is enabled to record the region of interest with a sufficient resolution. In such embodiment, the camera is automatically positioned without human interaction.

In step S03, the camera records one or more reference images of the person's region of interest without the manipulation tool being activated, i.e. without the region of interest being manipulated. Such one or more reference images may later on in the determination step S3 be used for being compared to the images taken by the camera while the manipulation tool is active. Hence, the images taken in step S03 serve as reference images and help identifying variations in the skin layout of the region of interest in response to the manipulation. After having taken the images absent any manipulation, the method continues with step S1, i.e. the activation of the manipulation tool.

FIG. 6 illustrates another flowchart representing a variant of a method according to an embodiment of the present invention. In this embodiment, the step S3 of determining the parameter of the skin in the region of interest is further elaborated. In a first step S31, the one or more reference images taken from the person's region of interest absent manipulation are analyzed. It is preferred, that in this step the region of interest is searched for features by means of a pattern recognition software. The features identified may be flagged, highlighted or otherwise noted in the reference images, as a result of the computation.

In a second step S32, the one or more images taken from the person's region of interest while being manipulated are analyzed. It is preferred, that in this step, by means of the pattern recognition software, the recorded images are searched for the very features that were identified in previous step S31 in the reference images. Any features recognized again may be flagged, highlighted or otherwise noted in the recorded images, as a result of the computation. Preferably, new features detected only in the recorded images but not in the reference images may be identified, flagged and/or highlighted. Also, features detected in the reference images but no longer in the recorded images may be identified flagged and/or highlighted.

In step S33, a comparison is made as to the features recaptured in the recorded images. Those features are quantified e.g. as to the distance of relocation or shift, which obviously was induced by the manipulation tool.

In step S34, the parameter such as the elasticity of the skin in the region of interest is determined from the results of the analysis in previous steps S34, and possibly also S33 and S32, when it comes to the inclusion of disappearing and/or appearing features. It is continued with step S4.

While there are shown and described presently preferred embodiments of the invention, it is to be distinctly understood that the invention is not limited thereto but may be otherwise variously embodied and practiced within the scope of the following claims.

1. Apparatus for determining a parameter of skin in a region of interest of a person's body, comprising
a camera arranged and configured to record images of the region of interest,
a control unit configured to determine the parameter of the skin in the region of interest by analyzing one or more images of the region of interest recorded by the camera while the region of interest is manipulated,
wherein the control unit is configured to, based on the determined parameter of skin, generate a cutting pattern with respect to the region of interest, either from scratch or by adapting an existing cutting pattern in preparation of a surgical intervention along the cutting pattern.

2. Apparatus for determining a parameter of skin in a region of interest of a person's body, comprising
a camera arranged and configured to record images of the region of interest,
a control unit configured to determine the parameter of the skin in the region of interest by analyzing one or more images of the region of interest recorded by the camera while the region of interest is manipulated,
a manipulation tool arranged and configured to manipulate the person's skin in the region of interest,
wherein the control unit is configured to determine the parameter of the skin in the region of interest by analyzing the one or more images of the region of interest recorded by the camera while the region of interest is manipulated by the manipulation tool,
wherein the parameter is elasticity,
wherein the manipulation tool is configured to one or more of shift, stretch, tighten and push together the skin in the region of interest,
wherein the manipulation tool comprises an air blower for generating an air jet directed at the region of interest for manipulating the person's skin in the region of interest, or wherein the manipulation tool comprises a mechanical structure for in-contact manipulating the person's skin in the region of interest,
wherein the manipulation tool comprises a mechanical structure for in-contact manipulating the person's skin in the region of interest.

3-5. (canceled)

6. Apparatus according to claim 2,
wherein the control unit is configured to control the manipulation tool, including one or more of:
positioning the manipulation tool;
activating the manipulation tool;
adjusting a defined power to drive the manipulation tool or adjusting the manipulation tool to exert a defined force; and
switching the manipulation tool between different states represented by different activation levels.

7. Apparatus according to claim 1,
wherein the control unit is configured to determine the parameter by means of comparing the one or more images recorded by the camera while the region of interest is manipulated with one or more reference images of the region of interest without the region of interest being manipulated,
wherein the control unit is configured to identify one or more features in the one or more reference images,
wherein the control unit is configured to identify the one or more features in the one or more recorded images,
wherein the control unit is configured to determine a distance between the one or more fea-tures identified in the one or more reference images and the one or more features identified in the one or more recorded images,
wherein the control unit is configured to determine the parameter subject to the determined distance.

8-9. (canceled)

10. Apparatus for determining a parameter of skin in a region of interest of a person's body, comprising
a camera arranged and configured to record images of the region of interest,
a control unit configured to determine the parameter of the skin in the region of interest by analyzing one or more images of the region of interest recorded by the camera while the region of interest is manipulated,
a manipulation tool arranged and configured to manipulate the person's skin in the region of interest,
wherein the control unit is configured to determine the parameter of the skin in the region of interest by analyzing the one or more images of the region of interest recorded by the camera while the region of interest is manipulated by the manipulation tool,
wherein the control unit is configured to determine the parameter subject to a defined power the manipulation tool is driven with or a defined force the manipulation tool is adjusted to exert.

11. Apparatus according to claim 1,
wherein the control unit is configured to determine the parameter subject to personal data including one or more of age of the person, gender of the person and origin of the person.

12. Apparatus according to claim 2,
wherein the control unit is configured to execute a computerized program including the steps of:
controlling the camera to take one or more reference images of the region of interest while the manipulation tool is deactivated,
activating the manipulation tool to manipulate the skin in the region of interest,
controlling the camera to record the one or more images of the region of interest while the manipulation tool is activated and manipulates the skin in the region of interest, and
determining the parameter by comparing the one or more reference images with the one or more recorded images.

13-18. (canceled)

19. Apparatus according to claim 1 comprising
an output unit for outputting the determined parameter,
in particular wherein the output unit is a display for illustrating the determined parameter,
in particular wherein the control unit is configured to display the one or more recorded images and/or the one or more reference images on the display,
in particular wherein the control unit is configured to display the parameter on the display in response to its determination,
in particular wherein the control unit is configured to display the parameter color coded on the display, in particular color-coded in the region of interest in the one or more recorded images and/or the one or more reference images displayed.

20. (canceled)

21. Apparatus according to claim 1,
comprising a light tool configured to project and/or write the cutting pattern onto the body of the person in the region of interest.

22. Apparatus according to claim 1,
comprising a cutting tool, in particular a laser, configured to cut the cutting pattern into the skin of the person in the region of interest.

23. Apparatus according to claim 1,
wherein the control unit is configured to computer control the cutting of the cutting pattern into the skin of the person in the region of interest.

24. Apparatus according to claim 1,
wherein the control unit is configured to display the cutting pattern in the region of inter-est of the one or more recorded images and/or the one or more reference images displayed.

25-27. (canceled)

28. Method for determining a parameter of skin in a region of interest of a person's body, comprising
manipulating the person's skin in the region of interest,
recording one or more images of the region of interest while the region of interest is manipulated (S2),
computerized determining the parameter of the skin in the region of interest by analyzing the recorded one or more images, and
computerized determining a cutting pattern with respect to the region of interest based on the determined parameter of skin, either from scratch or by adapting an existing cutting pattern, in preparation of a surgical intervention along the cutting pattern.

29. Method according to claim 28,
wherein the parameter is elasticity,
wherein the region of interest includes an eyelid,
wherein the cutting pattern is designated for conducting a lid lift in case of the region of interest being the eyelid.

30-33. (canceled)

34. Method according to claim 28, comprising
providing one or more reference images of the region of interest taken without the person's skin in the region of interest being manipulated, and
determining the parameter by comparing the one or more reference images with the one or more recorded images,
wherein the determination step includes:
identifying one or more features in the one or more reference images,
identifying the one or more features in the one or more recorded images,
determining a distance between the one or more features identified in the one or more reference images and the one or more features identified in the one or more recorded images, and
determining the parameter subject to the determined distance.

35-39. (canceled)

40. Method according to claim 28, comprising projecting and/or writing the cutting pattern onto the region of interest of the person by means of a light tool.

41. Method according to claim 28, comprising
cutting, by means of a computer controlled cutting tool, preferably by means of a computer con-trolled laser, the cutting pattern into the skin of the person in the region of interest.

42. Method according to claim 28, comprising
displaying the one or more of the recorded images and/or the reference images and displaying the cutting pattern in the region of interest on the displayed one or more images.

43. Method according to claim 28, comprising
submitting a pre-op image of the region of interest and an envisaged cutting pattern to an artifi-cial neural network, and
in response to the submission receiving a post-op image of the region of interest from the artificial neural network.

44. Computer program element, comprising computer program code means configured to perform a method according to claim 28 when executed on a control unit.
